(19) United States
(12) Patent Application Publication
(10) Pub. No.: US 2002/0034817 A1
(43) Pub. Date: Mar. 21, 2002

(54) Process and apparatus for isolating and continuosly cultivating, harvesting, and processing of a substantially pure form of a desired species of algae

(76) Inventors: Eric C. Henry, Corvallis, OR (US); Kenneth L. Cordrey, Salem, OR (US); Christopher A. Bakken, Beaverton, OR (US); Craig L. Berkman, Portland, OR (US)

Correspondence Address: Mr. Lynn G. Foster, Foster & Foster, LLC, 602 E. 300 S., Salt Lake City, UT, 84102, US

(21) Appl. No.: 09/971,992

(22) Filed: Oct. 5, 2001

Related U.S. Application Data
(62) Division of application No. 09/431,855, filed on Nov. 2, 1999.
Application No. 09/431,855 is a division of application No. 09/106,195, filed on Jun. 26, 1998.

(51) Int. Cl. C12N 1/12

(52) U.S. Cl. 435/257.1; 435/261

(57) ABSTRACT

Novel closed system methods and apparatus for the production and utilization of algae are disclosed. A substantially pure form of a desired strain of alga is obtained and cultivated (or isolated and grown). The desired species of alga is isolated from the contaminants and other algae and placed in the controlled environment where its growth is cultivated without contaminants. At desired points in time, a portion of the cultivated alga is removed, with the remainder serving as progenitor stock for growing more of the desired alga. The removed alga is processed and placed in product form.

### FIELD OF INVENTION

The present invention relates to novel processes and apparatus by which a desired naturally-occurring species of algae is isolated in a substantially pure form from other co-mingled naturally-occurring species of algae, among other things, and thereafter continuously cultivated, without introduction of contaminants, so that the quantity thereof multiplies or enlarges, without seasonal variations, while remaining substantially pure. Part of the cultivated alga is harvested leaving a residual portion to further multiply thereby replenishing that which is harvested by its continued growth. The harvested portion of the isolated and substantially pure selected species of algae is processed, including at least drying and packaged for use, usually in a powdered form, all without introduction of contamination.

### BACKGROUND

In the past, several species of algae have been collectively recovered and processed, in an impure condition, resulting in end product that contains both contaminants and a plurality of dried algae.

The recovery, in the past, has comprised skimming, screening, filtering, centrifuging or flocculating all algae species and accompanying impurities from a naturally-occurring lake or a man-made or naturally-occurring pond, or below a weir over which water and the intermingled algae flows as effluent from the lake, pond or river, for example. Because of seasonal variation in algae reproduction in lakes, ponds and streams, for example, significant quantities of algae are not available at all times for harvesting.

The prior art has not encompassed isolation and man-directed cultivation in a closed system or controlled environment of a single desired species. It also has not encompassed large-scale controlled, continuous growth of a single species of alga in protected or enclosed environment from which a substantially pure form of the single species of algae is derived, processed, dried, and packaged. The prior art has also not encompassed technology by which availability of algae is not affected by weather conditions or seasonal cycles. Large-scale commercial production of microalgae typically is by photosynthesis in open ponds. One exception is known to exist, namely heterotrophic closed system production of algae. Cyanotech and possibly others are supplying phycobiliproteins that may be produced by algae in closed systems, but the quantities produced are undoubtedly small because the market (laboratory fine chemicals) is small.

All prior commercial production of Aphanizomenon flos-aquae (AFA) has been from Upper Klamath Lake, Oreg., and is part of a naturally-occurring and mixed multi-algal biomass. Upper Klamath Lake is a very shallow body of water with an average depth of less than three (3) meters. It is fed from surface springs, three small rivers, and a small number of geothermal volcanic vents. The lake is subjected to pollution by agricultural runoff as well as massive larval hatches of insects (midges) that contaminate any algae harvested.

The algal mix harvested normally includes various species of green algae and blue-green algae. During certain months of the year (typically from July to October) blue-green algal species are predominant. Some of the non-AFA species of blue-green algae present are known to produce dangerous hepatotoxins and neurotoxins. Although AFA in monoalgal culture has been cultivated in laboratory-scale closed systems on a research only basis, there has been no prior commercial-scale monoalgal cultivation.

The other species of microalgae that are photosynthetically cultivated for commercial production, such as Spirulina, Dunaliella, Chlorella, and Haematococcus, are all produced in a collective fashion in open natural or man-made ponds that are subject to contamination by air pollution, wind-borne dust and debris, insects, and birds, as well as invasion by undesirable species of algae, fungi, and other aquatic organisms.

### BRIEF SUMMARY AND OBJECT OF THE INVENTION

In brief summary, the present invention overcomes or alleviates past problems associated with the production and utilization of algae. The present invention provides novel methodology and apparatus by which a substantially pure form of a desired strain of alga is obtained and cultivated (or isolated and grown). The technology is usually in the form of a closed system. The technology of this invention is not dependent upon weather conditions and/or seasonal cycles. The initial source of supply may be a fresh water lake or other source where the desired strain of alga is found in the company of impurities or contaminants and other species of algae, among other things. The desired species of alga is isolated from the contaminants and other algae and placed in a controlled environment where it is grown without contaminants. At desired points in time, a portion of the cultivated alga is removed, with the remainder serving as progenitor stock from which the supply of the desired alga is regenerated. The removed and substantially pure, single species of alga is processed, including but not necessarily limited to drying, and packaging. The processing may comprise use of a filler which adds body to the light powder comprising the dried alga. The packaging can be in capsules or tablets in bottles, for example.

With the foregoing in mind, it is a primary object of the present invention to overcome or alleviate problems of the prior art associated with the production and utilization of algae.

It is another major object of the present invention to provide novel methodology and apparatus by which algae are recovered for utilization.

Another paramount object is the provision of novel apparatus and processes by which a desired strain of alga is isolated for controlled production and use.

A further important object is the provision of novel methodology and apparatus by which a substantially pure form of a desired strain of alga is obtained.

It is a significant object of the present invention to utilize a naturally-occurring source of algae from which a desired strain of alga is isolated substantially free from impurities and contaminants.

It is an object of significance to provide apparatus and processes for the production of algae, which are not limited by weather, seasonal changes and/or harvest cycles.

It is an object of value to provide novel processes and apparatus by which growth of a desired strain of alga is cultivated in a controlled environment substantially free from contaminants, harvested, dried, processed, and packaged.

A further object is the provision of novel methodology and apparatus by which an alga is continuously grown in a substantially pure form, with part of the alga being harvested and processed, including packaging for subsequent use, while a progenitor stock of growing alga is retained in the growth mode to replenish the supply.

It is an object of paramount value to provide novel apparatus and processes for incubation-type production of algae, which are significantly better and more efficient than natural harvesting techniques.

It is a further object of importance to provide novel algae.

Another valuable object is the cultivation and harvesting of a desired alga, using closed system apparatus and methodology.

These and other objects and features of the present invention will be apparent from the detailed description taken with reference to the accompanying drawings.

### BRIEF DESCRIPTION OF THE DRAWINGS

FIG. 1 is a pictorial sketch of one manner by which an initial sample, comprising multiple species of algae and contaminants, is obtained from a pond, river or other body of water;

FIG. 2 is a diagrammatic representation of the acts taken in one process by which a single species of alga in a pure environment is obtained from a sample of the type acquired using any technique such as that depicted in FIG. 1;

FIG. 3 is a diagrammatic representation of one series of acts comprising use of a multi-well plate by which one or more cells or filaments of a single isolated alga in a medium free of all other organisms is propagated or caused to multiply for the purposes of later being bifurcated or separated into (1) a portion comprising stock for repeating the propagation process and (2) a second harvested portion, which is further propagated in mass culture and eventually processed into a commercially available product placed in marketable and usable form;

FIG. 4 is a diagrammatic representation of a series of acts comprising use of jugs or large containers by which a single isolated alga in a pure environment is propagated or caused to multiply for the purposes of later being bifurcated or separated into (1) a portion comprising stock for repeating the propagation process and (2) a second harvested portion, which is further processed into a commercially available product placed in marketable and usable form;

FIG. 5 is a block diagram of a computer system for monitoring and controlling the conditions of a culture of alga and growth media;

FIG. 6 is a block diagram of one way by which the present invention may be practiced;

FIG. 7 is a block diagram of another way by which the present invention may be practiced;

FIG. 8 is a block diagram of a further way by which the present invention may be practiced;

FIG. 9 is a block diagram of one more way by which the present invention may be practiced;

FIG. 10 is a schematic cross-section taken along lines 10-10 of FIG. 9;

FIG. 11 is a schematic in cross-section showing certain controls for use with the embodiment of FIG. 9;

FIG. 12 is a perspective of one embodiment of an array of light-transmitting coils of tubes used to grow the alga, and sources of artificial light, together with cylindrical support structure upon which the tubes are coiled and the light sources are mounted, respectively;

FIG. 13 is an enlarged fragmentary perspective from the inside of the cylindrical coil support structure of FIG. 12;

FIG. 14 is a vertical cross-section taken along lines 14-14 of FIG. 13;

FIG. 15 is a diagrammatic illustration of a sparging tank, shown in cross-section;

FIG. 16 is a diagrammatic illustration of a sparging manifold, shown in cross-section;

FIG. 17 is a perspective of a collection tank;

FIG. 18 is a flow chart illustrating one way in which pig cleaning can be achieved;

FIG. 19 is a perspective of an algal growth tank;

FIG. 20 is a flow chart of a system for diverting, catching and removing a pig after a cleaning cycle; and

FIG. 21 is a diagram of a pig insertion system.

### DETAILED DESCRIPTION OF THE ILLUSTRATED EMBODIMENT

### Definitions

The following definitions appropriately apply and are adopted for purposes of this specification:

alga: a plant of the group Algae or of the divisions or classes including Chlorophyceae, Euglenophyceae, Pyrrophyceae, Chrysophyceae, Phaeophyceae, Cyanophyceae, and Rhodophyceae.

algae: in some classifications: a major group of lower plants that is often included in Thallophyta, that comprises usu. photosynthetic plants of extremely varied morphology and physiology, and that is now commonly considered to be a heterogeneous assemblage.

algal: relating to, consisting of, or resembling an alga or algae.

### An Overview of the Present Methodology

Unlike the recovery and propagation of multiple species mass cultivation of impure algae of the prior art, the present invention embraces isolation or separation or creation of one desired strain or species of naturally-occurring alga from other algae and other growing organisms and from impurities and contaminants, heretofore commingled with the desired alga. In appropriate circumstances where a pure composite product is desired, two or more species of algae may be cultivated, harvested and reduced to product form using principles of the present invention. The present methodology is not materially influenced by seasonal changes nor by variations in the weather. The process remains contamination free. The isolated alga of the desired species may be genetically unique AFA (Aphanizomenon flos-aquae), or Oocystis borgei and/or other species of the genus Haematococcus or class Eustigmatophyceae amongst others. AFA is best grown with moderate light, is sensitive to nutrient depletion and shear, and is very sensitive to light and aeration. Oocystis is best grown with high light intensities and tolerates nutrient depletion and pH extremes.

The separated substantially pure alga is cultivated or caused to multiply, grow, or propagate in a controlled environment and a substantially pure state, using appropriate nutrients, light, and temperature. Some if not all forms of artificial light are believed to economically enhance algae growth, some more than others. It has been found that isolation and propagation of the alga is optimized by dispersing or separating the cells or filaments that comprise the alga. One way of isolation is to place a single cell or filament in each well of a multi-well plate in which a culture medium has been placed. In the case of the AFA alga, for example, the culture medium is without fixed nitrogen and is held at normal room temperature, which accommodates growth of AFA while inhibiting growth of most other algae and microbes. One or more trace elements, such as molybdenum, may be added for nutritional purposes as deemed appropriate by those skilled in the art. After a few days, when certain wells have been found to contain new algal growth without associated contaminants, the pure alga is transferred to a small cylinder or other container filled with nutritional media.

In small scale production/propagation apparatus, an aeration tube, of fluoropolymer, or other suitable device is used to introduce air into the growth medium and growing algal culture carried, for example, in a tube. The alga typically exhibits itself under such conditions as ideally single cells or filaments of numerous cells, but they may also be as small agglomerations of cells either singly or as several filaments, often referred to as flakes. Aeration takes place in the form of air bubbles, preferably small ones. These bubbles are discharged into this alga-bearing medium, which stirs the mixture to homogenize and agitate it. Trace amounts of CO₂ are normally contained within the compressed air. CO₂ is required for photosynthesis in the presence of light, by which sugar nutrients are derived for algal growth accompanied by the release of O₂O. The aeration also drives off excess O₂ and odor-producing volatiles.

The pH is controlled so as to be maintained at a suitable value to prevent physiological shock to the alga and to stimulate effective photosynthesis.

As mentioned, in lieu of wells in a well plate, larger cultivation reservoirs can be used. For example, clear or blue tinted 5-gallon containers (carboys), formed of synthetic resinous material comprised of polycarbonate, may be used. The top of each container is capped to form a closed system which is equipped to accommodate influent air, for aeration, under slightly positive pressure and to vent O₂ derived during photosynthesis.

In circumstances where microbes are of concern, use of filters and/or antibiotics to eliminate the microbes is appropriate, as will be readily apparent to those possessed of skill in the art.

Adequate algal growth in the carboys may require an extended time, perhaps several weeks, following which the carboys can be either entirely or partially emptied into a container for further processing. When entirely emptied, the carboys are washed, sterilized and restocked with the desired alga and the medium. When the carboys are partially emptied, the remainder of the desired alga and medium form a stock for further growing of another crop of the alga.

If optimum propagation of the alga occurs before it is desired to harvest the alga, the alga-containing carboys can be placed in a dark environment and/or nutrients withheld, which will curtail further growth.

Larger closed systems may also be used for large-scale continuous commercial production of a desired single species of alga.

Toxin production is typically avoided by initial selection of non-toxic strains. Genetic engineering may be applied to eliminate or greatly alleviate production of toxins by a selected blue-green alga.

The isolation of one alga could take place after group cultivation of several species of algae.

The potential for the introduction of contamination is avoided during, for example, isolation of the selected strain of alga, cell or filament separation, introduction of the alga into a nutrient-containing medium, containerizing of the alga, the introduction of CO₂-containing air, the venting of O₂ derived from photosynthesis, the removal of the cultivated alga, the drying of the alga, and the mixing of additives, if any, with the powdered alga, and packaging.

While the contrary is preferable, antibiotics and/or anti-microbial substances may be used to kill residual bacteria, fungi, protozoa, yeast and the like. If done, this preferably takes place immediately after isolation of the specimen obtained from a naturally-occurring body of water.

### Source of Root Stock and Isolation of Selected Algae

As mentioned above, a root stock of AFA (Aphanizomenon flos-aquae), Oocystis borgei or other species of the genus Haematococcus or class Eustigmatophyceae amongst others may be obtained through isolation of the desired species after a multiple-species impure sample is drawn from Klamath Lake in Oregon or from some other fresh water or marine locality.

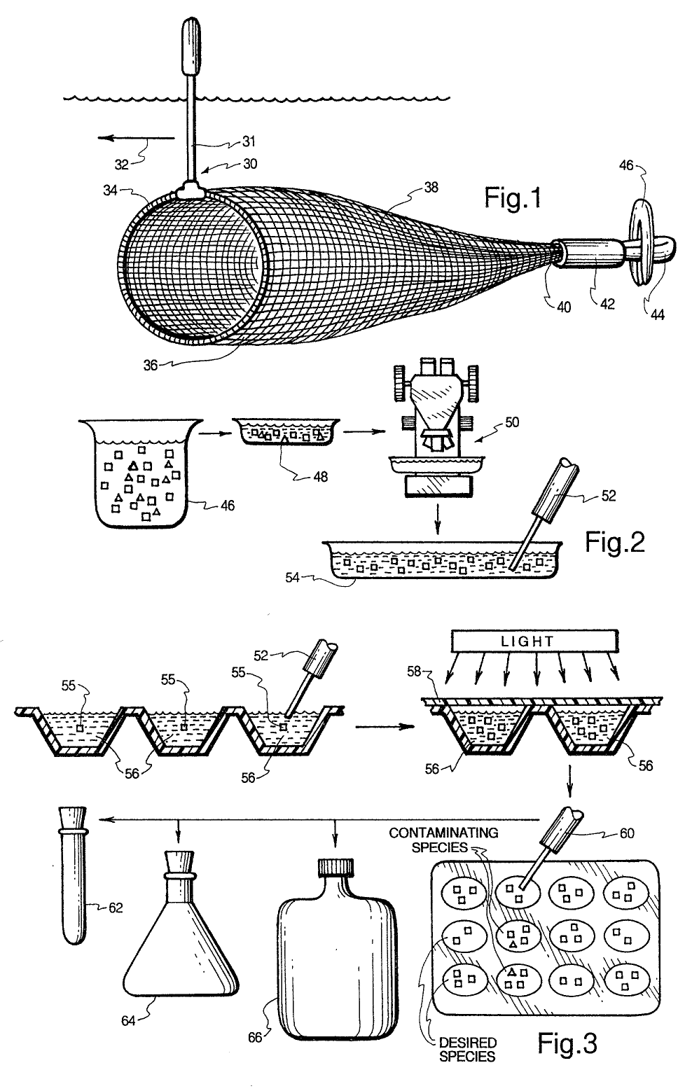

In reference to FIG. 1, typically a handle-held plankton net device, generally designated 30,is manually moved through the algae-containing pond by force applied at handle 31 in the direction of arrow 32. The handle 31 merges with a rigid loop 34, to which one end 36 of a conical netting comprised of fine mesh 38 is attached.

The trailing end 40 of the mesh 38 is connected to a transparent rigid plastic tube 42, which in turn is connected to a length of flexible tubing 44, which is held in a normally-closed position by a spring clamp 46.

When the sample has been so positioned in tube 42, the device is removed from the pond, while rotating the handle 31 from the vertical to a horizontal position. The multi-algae sample in tube 42, shown as rectangular and triangular flakes, is rained by gravity into a suitable container (such as beaker 48, shown in FIG. 2) by opening the clamp 46. Other species of algae can also be obtained in the same way.

Analyses by microscopy of the AFA obtained from Klamath Lake root stock and from AFA products on the market demonstrates that the algae products derived from the lake, using conventional wisdom, contain several species of algae, in addition to certain contaminants, such as insects, crustaceans, feathers, and other debris and filth. The same may be true of any other algal species that is obtained from ponds or other growth facilities that are not isolated from open air.

Numerous species of algae grow collectively and intermingle in naturally-occurring sources of algae. Therefore, it is necessary, after gaining a sample from the lake or other source, to separate the desired single species for isolated cultivation thereof in an environment that avoids contamination or other undesirable properties, the source of which may be other species.

In the past, it has been impractical if not impossible for commercial processors to separate and remove a particular species (Microcystis aeruginosa) which has been shown to produce potent hepatotoxins (microcystins) from the other algae harvested from Klamath Lake. Thus, algae producers have produced algae products each comprised of several algae. Potentially harmful toxins (including hepatotoxins and neurotoxins) may be present in a multiple-algae environment and multiple-algae products.

For sparging and other purposes, compressed and filtered air is used. Filters used for compressed air remove oil, water, and other materials down to 0.1 micrometers in size. Therefore, any microorganisms in the air line from the compressor are removed during filtration. CO₂ may be added to the air stream or may be bubbled separately. The amount of CO₂ gas added is controlled by monitoring the pH. Algal photosynthesis consumes CO₂, which causes the pH to rise. An inordinately high pH is detrimental to the health of the algal culture. The addition of CO₂ gas to augment that provided by normal aeration from sparging causes the formation of carboxylic acid, which brings the pH down into a more optimal range. Additionally, the CO₂ gas serves as a carbon source for photosynthesis. Sparging with streams of dispersed small bubbles serves the purpose of inducing rapid diffusion and gas exchange.

The present process for separating any one of AFA, Oocystis borgei or other species of the genus Haematococcus or class Eustigmatophyceae, amongst others, from the other species found in the root stock is described below, in reference to FIGS. 2 through 4.

Samples may be collected from bodies of water, such as a puddle, pool, pond, lake, river or ocean, as described in respect to FIG. 1. Restated, water may be sampled with a conical plankton net 30, by which the algae are concentrated into a transparent tube 42 attached to the tip of the net. The concentrated sample is removed from the tube 42 through the bottom thereof, to which is attached a section of flexible plastic or rubber tubing 44 closed by a clamp 46; when the clamp is released, the sample that flows out is directed into a collecting container. For sampling shallow bodies of water, a small net typically of six (6) inches or less maximum diameter is used. Rather than the conventional practice of suspending the net by three lines attached to the circular frame that holds the mouth of the net open, the frame is instead attached to a pole or handle 31, which is several feet long so that the net can be more precisely moved and oriented in the shallow water. In this way, obstructions are avoided and desired small patches of the habitat can be more accurately sampled.

Conspicuous aggregations of algae may be sampled directly by drawing up water with a turkey baster or a disposable polyethylene pipet. Samples may be transferred to a collecting bottle or vial. A polyethylene pipet containing a sample may be sealed at the tip with a flame, preventing spilling or contamination of the sample.

Floating films of algae may be sampled by laying a sheet of paper such as newsprint on the floating film, causing the film to adhere to the paper, then raising the paper and folding it so that the film lies inside the fold. The paper can then be sealed in a bottle or small plastic bag, such as the “self-sealing” type that may be closed with an integral zipper or wire.

Aggregations of algae on surfaces such as rocks and plants may be sampled by scraping the algae from the surface with a stainless steel knife, then transferring the scrapings to a collecting bottle, vial, or bag with forceps or pipet; or by collecting pieces of the rock or plant bearing the algae. Thus, in addition to aquatic habitats, algae may also be sampled from terrestrial surfaces such as soil, rocks and trees, by scraping surfaces or taking samples of the substrates bearing the algae.

Samples of algae may be transported in an insulated container, which may be cooled by ice or thermoelectric refrigeration, sometimes operated from the 12V DC power of an automobile. Large water samples of several liters containing concentrated algal biomass may require aeration to supply oxygen, supplied by vibratory air pumps that in an automobile can be powered from a 12V DC-to-120V AC inverter.

In the laboratory, as shown diagrammatically in FIG. 2, field samples at 46 containing mixed algal species, bacteria, etc. may be separated into smaller subsamples. If a subsample, at container 48, is dense, it is diluted. A desired subsample is examined under a microscope, such as a stereo-microscope, for the presence of desired species. Initial examination and manipulation under a binocular dissection microscope, at 50, can remove a great part of the several unwanted species from the sample. Individual algal cells may be isolated within the subsample under the highest magnification of a good-quality dissection microscope; smaller species may readily be manipulated with the aid of an inverted microscope.

Algal cells, especially those adhering to hard surfaces or larger species, may be separated from the substrates by violent shaking in a small (ca. 1 mL) vial ½-¾ filled with water, by use of a “vortex” mixer or dental amalgam-type mixer.

The single cells or filaments of the desired alga, isolated within the subsample are removed with a fine needle or pipet 52, for example, and, thereafter, transferred to a small volume of appropriate culture medium in a well of a multi-well plate, small petri dish, or other suitable container 54. A fine (30 gauge) hypodermic needle or broken-off tip of a fine glass pipet may be used as a knife to sever a short segment of a multicellular filament, which can then be transferred to a separate vessel.

### The Growth Media and Control Thereof

Careful formulation of a medium from among a large number of possible ingredients that can affect alga growth is desirable. The composition of the medium may vary widely depending on the specific alga being grown and other factors. Typical ingredients are selected from those listed below in the Examples. Likewise, frequent chemical analysis of the medium as the culture grows is also desirable, but may not be an essential criterion.

In the case of AFA, the culture may be started with a phosphate concentration as much as ten to twenty times the usual level, the usual level being 100 to 200 μM. Even though this is counter to current wisdom as to the concentration, the performance of the cultures to date has been excellent.

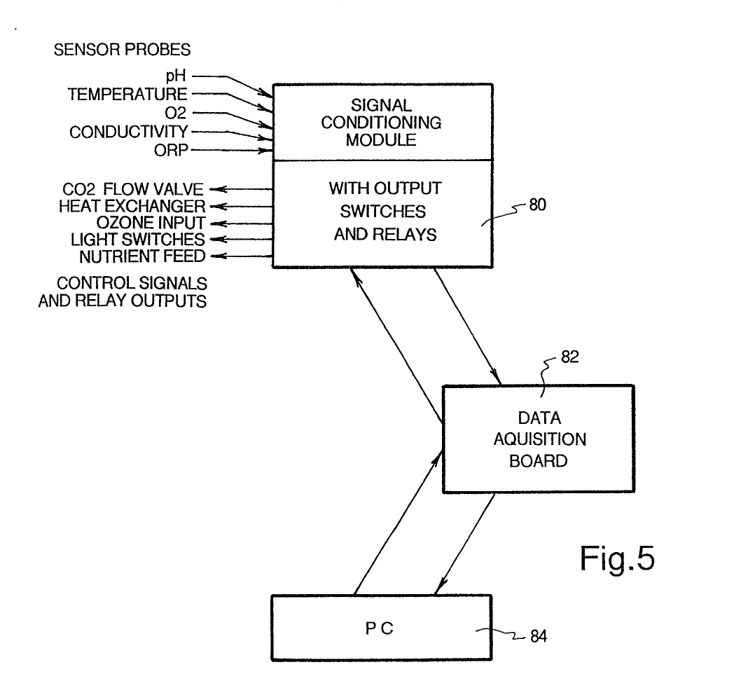

A conventional computer-based data acquisition and process control system may be used to both monitor and control culture conditions continuously (and remotely by modem). Computer control of culture conditions greatly facilitates control of fixed nitrogen, pH, carbon dioxide, and bicarbonate concentrations, which involve interactions that can cause deleterious instabilities in culture conditions, unless detected and corrected promptly. A block diagram for such a computer monitoring system is shown in FIG. 5.

Control of light intensity is important in the development of high-density cultures. For uninterrupted growth of the selected alga, artificial light is essential, although naturally-occurring light may be used in conjunction with artificial light. Growth of the culture is curtailed by depriving it of light.

For exemplary purposes only, the following are examples of conventional media, with concentrations in micromoles per liter:

### MEDIUM EXAMPLE NO. 1

ASM (Gorham et. al 1964; Verh. int. Verein. theor. angew. Limnol. 15:796-804)

| | |
|---|---|
| NaNO₃ | 1000 |
| MgSO4 | 200 |
| MgCl₂ | 200 |
| CaCl₂ | 100 |
| K₂HPO₄ | 100 |
| FeCl₃ | 2 |
| H₃BO₃ | 10 |
| MnCl₂ | 7 |
| ZnCl₂ | 0.8 |
| CoCl₂ | 0.02 |
| CuCl₂ | 0.0002 |
| Na₂EDTA | 20 |

### MEDIUM EXAMPLE NO. 2

Bold's Basal Medium (Nichols and Bold 1964; J. Phycol 1:34-8)

| | |
|---|---|
| K₂HPO₄ | 430 |
| KH₂PO₄ | 1290 |
| NaNO₃ | 2940 |
| NaCl | 430 |
| MgSO₄ | 300 |
| CaCl₂ | 170 |
| H₃BO₃ | 184.7 |
| EDTA | 171 |
| Co(NO₃)₂ | 16.8 |
| CuSO₄ | 62.9 |
| FeSO₄ | 17.9 |
| MoO₃ | 4.9 |
| MnCl₂ | 7.3 |
| ZnSO₄ | 30.7 |
| KOH | 553 |

### MEDIUM EXAMPLE NO. 3

Chu “No. 10” (Chu 1942; J. Ecol. 30:284-325)

| | |
|---|---|
| K₂HPO₄ | 60 |
| Na₂CO₃ | 190 |
| Na₂SiO₃ | 200 |
| MgSO₄ | 100 |
| Ca(NO₃)₂ | 240 |
| FeCl₃ | 4.9 |

### MEDIUM EXAMPLE NO. 4

Waris (Waris 1953; Physiol. Plant. 6:538-43)

| | |
|---|---|
| KNO₃ | 990 |
| MgSO₄ | 80 |
| CaSO₄ | 370 |
| EDTA | 17.86 |
| FeSO₄ | 17.9 |
| KOH | 54 |

The composition of the presently preferred medium for initial propagation of AFA in small volumes is:

| | |
|---|---|
| NH₄H₂PO₄ | 2000 |
| MgSO4 | 200 |
| MgCl₂ | 200 |
| CaCl₂ | 100 |
| FeCl₃ | 4 |
| H₃BO₃ | 40 |
| MnCl₂ | 7 |
| ZnCl₂ | 3.2 |
| CoCl₂ | 0.08 |
| CuCl₂ | 0.0008 |
| NaMoO₄ | 0.1 |
| Na₂EDTA | 20 |

This medium employs NH₄H₂PO₄ as a source of both nitrogen and phosphate.

The composition of the presently preferred medium for propagating AFA in carboys is:

| | |
|---|---|
| NH₄Cl | 2000 |
| MgSO4 | 200 |
| MgCl₂ | 200 |
| CaCl₂ | 100 |
| K₂HPO₄ | 200 |
| FeCl₃ | 4 |
| H₃BO₃ | 40 |
| MnCl₂ | 7 |
| ZnCl₂ | 3.2 |
| CoCl₂ | 0.08 |
| CuCl₂ | 0.0008 |
| NaMoO₄ | 0.1 |
| Na₂EDTA | 20 |

This medium employs NH₄Cl as the primary nitrogen source with NaNO₃ as backup.

The composition of the presently preferred medium for mass culture of AFA is:

| | |
|---|---|
| NaNO₃ | 2000 |
| MgSO4 | 200 |
| MgCl₂ | 200 |
| CaCl₂ | 100 |
| K₂HPO₄ | 200 |
| FeCl₃ | 4 |
| H₃BO₃ | 40 |
| MnCl₂ | 7 |
| ZnCl₂ | 3.2 |
| CoCl₂ | 0.08 |
| CuCl₂ | 0.0008 |
| NaMoO₄ | 0.1 |
| Na₂EDTA | 20 |

In reference to FIG. 3, where initial cultivated growth of the selected alga is via wells in a multiple well plate, the single cell or filament 55 of the desired alga is placed in each well 56 via a fine needle or pipet 52 together with a suitable medium comprising nutrients. The wells are sealed against contamination by a lid or cover 58. Incubation in each well 56 takes place in the presence of light to propagate the alga.

With the cover 58 removed, after sufficient algal density is achieved, microscopic analysis is used to determine wells having a contaminating species, shown as triangular flakes, and wells with the desired species only, shown as rectangular flakes. The material comprising a contaminating species is discarded while the material in the wells comprising only the desired species are used as source material for further algal cultivation. Specifically, material comprising only the desired species is transferred via sterile pipet or other suitable instrument to a larger container, such as test tube 62, flask 64 and/or vial or jug 66.

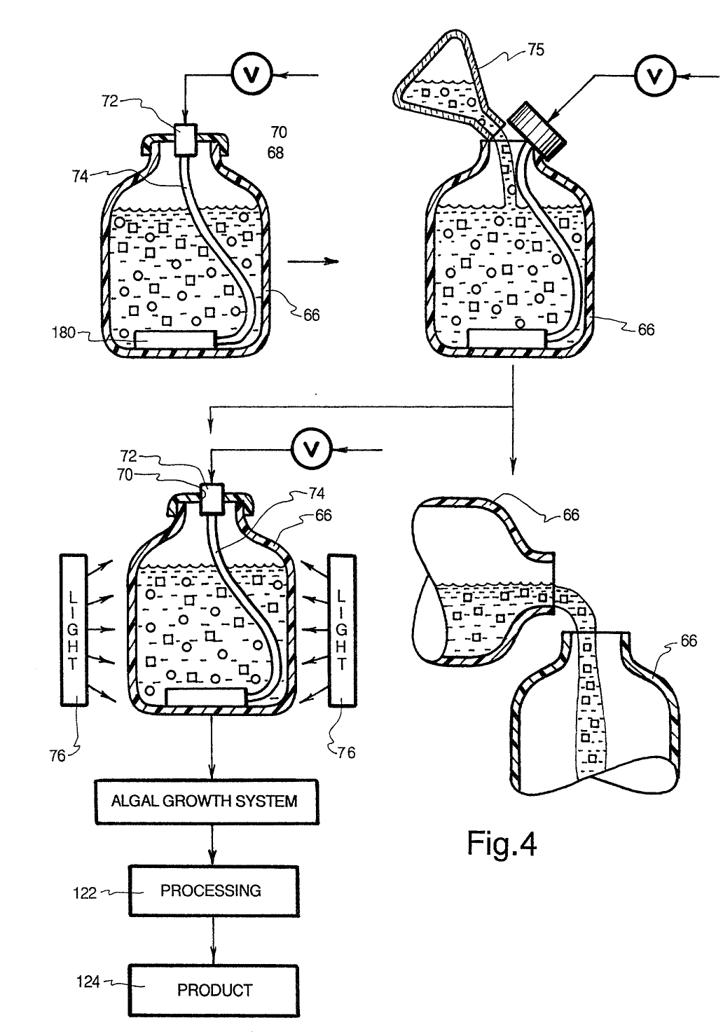

When a jug or carboy 66 is used, preferably it is formed of suitable synthetic resinous food grade material and is capped at 68 after the culture is added via a beaker 75, for example, together with a supply of liquid nutrients. See FIG. 4. The cap 68 comprises a central aperture 70 in which a porous plug 72 is positioned. An air line 74 passes snugly through the plug 72 delivering compressed air and CO₂ to the inside of carboy 66 in desired amounts and at a desired rate to support photosynthesis and sparging of O₂ in the presence of light at one or more sites 76. The compressed air and CO₂ are bubbled from a sparging rod 180 at the bottom of the carboy 66. Air and sparged O₂ collected at the top of the carboy 66 is discharged through plug 72, which may comprise a suitable foam material preventing entry of contaminants, including microorganisms, while allowing release to the atmosphere of compressed air and sparged O₂. The bubbles of sparging air and CO₂ also mix the alga and nutrient liquid in the carboy 66. When culture density increases sufficiently, it may be processed into a commercial product. Alternatively, when culture density increases sufficiently, part of the volume can be transferred to additional carboys, and medium can be added to the original carboy to restore full volume.

### Hydro Photo Cell Systems

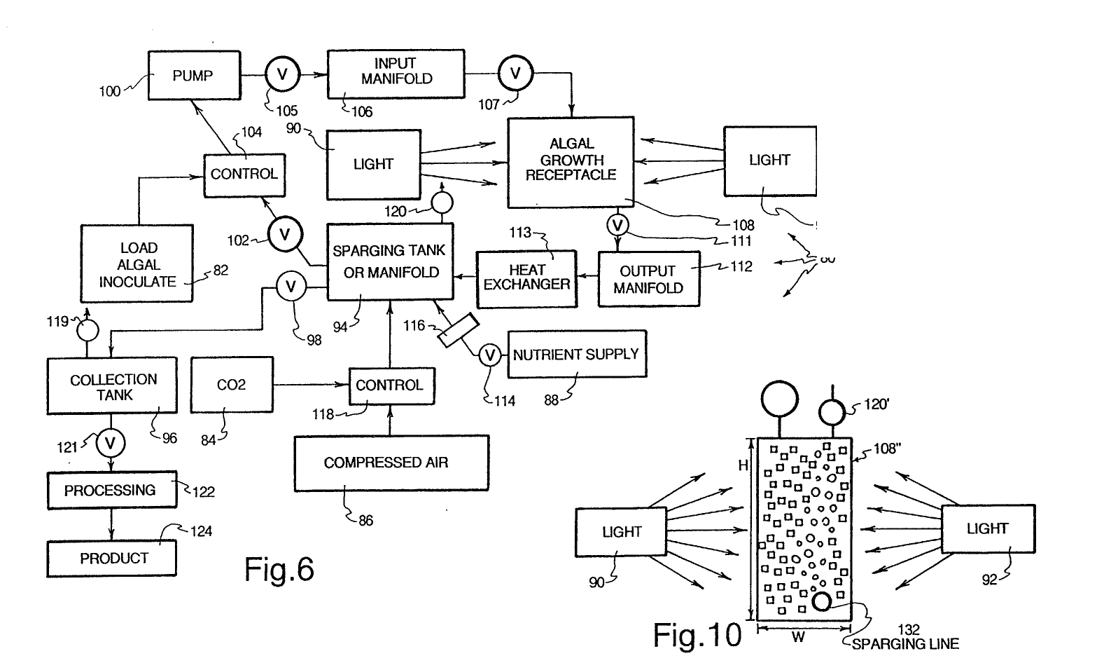

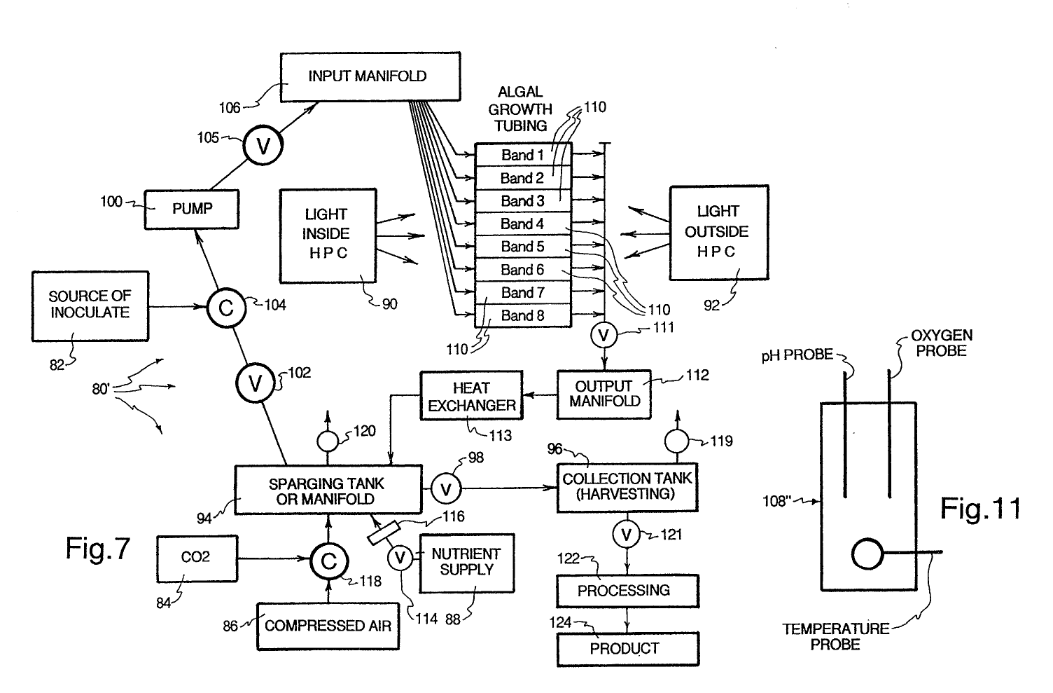

Representative hydro photo cell systems, which embrace the present invention are schematically depicted in FIGS. 6 and 7, which are generally designated 80, 80′, 130 and 130′, respectively. Many parts of system 80 are also comprised in the other systems 80′, 130 and 130′ and, to avoid duplication, each part will be described only once.

For growth of the desired isolated alga (AFA, for example) in enlarged or commercial scale quantities, Hydro Photo Cell system (HPC) 80 and/or HPC 80′ may be utilized. Basically, certain source sites are provided, i.e., a source 82 of the desired alga, a source 84 of carbon dioxide under pressure, a source 86 of air under pressure, a source 88 of nutrition for the alga in question and opposed sources 90 and 92 of light.

The systems 80 and 80′ are continuously circulating closed systems where liquid, comprising the selected alga and nutrients, is recirculated through several stations causing the alga to grow. A measured amount of liquid comprising concentrated alga is continuously displaced from a sparging tank (or manifold) 94 to a collection tank 96, across a valve 98. The remainder of the liquid comprising concentrated alga and residual nutrients in sparging tank 94 is displaced from tank 94, under force of pump 100, across valve 102 and control 104 through pump 100, across valve 105 to an input manifold 106.

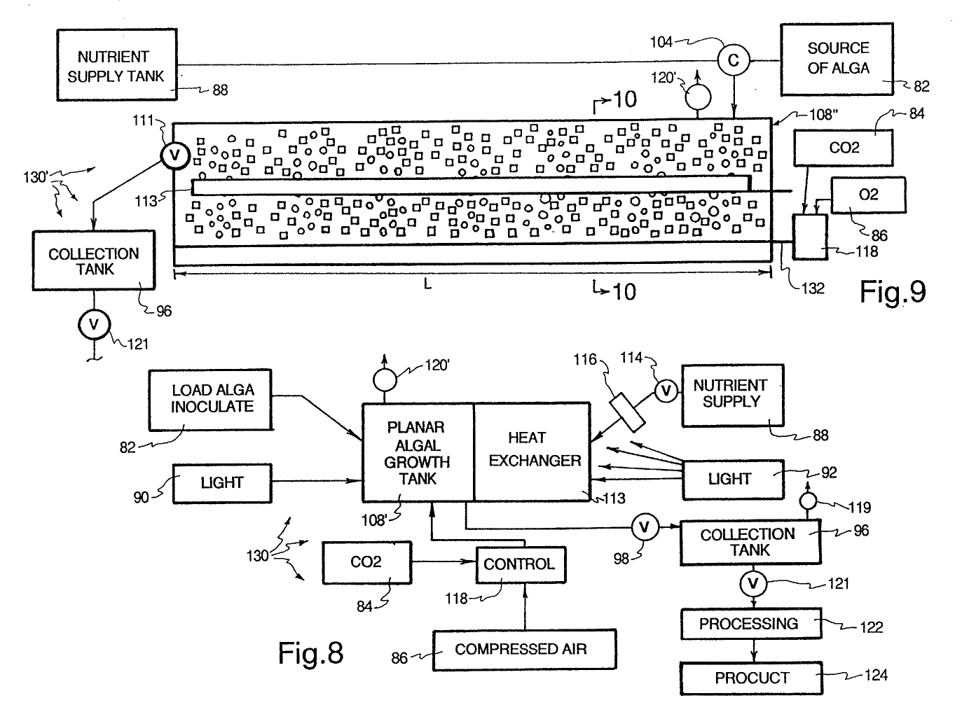

The positive pressure of the pump 100 drives the liquid through the input manifold 106 so as to displace the single influent stream into any suitable light-transmitting algal growth receptacle or receptacles 108 (FIG. 8), which may be an array of transparent tubes 110 (FIG. 9), which respectively receive a subdivided portion of the influent stream delivered to the input manifold 106. Each tube 110 has a predetermined diameter and length to control the dwell time of the liquid while therein to prevent toxicity, as explained hereinafter in greater detail.

As the liquid is displaced along each tube 110, the liquid is exposed substantially uniformly to oppositely directed light from light sources 90 and 92. Thus, photosynthesis efficiently takes place in each tube 110, which causes release of oxygen into the liquid.

In respect to FIG. 6, the continuous effluent from alga growth receptacle 108 may be displaced across valve 111 to an output manifold 112.

In addition to the circulated and recirculated liquid comprising alga and nutrients (media), a predetermined amount of the nutrients or medium is added periodically or continuously from source 88 across control 114. Typically, to kill microorganisms, the supply of nutrients added to tank 94 from source 88 is subjected to ultraviolet light at site 116. Also, suitable amounts of compressed air and carbon dioxide are added to the sparging tank 94 across control 118, typically as bubbles.

The compressed air serves to drive off or sparge excess dissolved oxygen in the liquid in receptacle 108 produced during photosynthesis. Thus, the oxygen is displaced to the top of tank 94, where it is released to the atmosphere via relief valve or vent 120. To maintain the desired liquid temperature, a heat exchanger 113 may receive liquid from the output manifold before discharging it to the tank 94. See FIGS. 6 and 7.

Liquid in collection tank 96, containing a high concentration of alga, is removed and processed, as indicated diagrammatically at site 122 in FIGS. 6 through 11, in a manner later described herein in greater detail, to produce the desired product, indicated diagrammatically at site 124.

Reference is now made specifically to FIGS. 8 through 11, which diagrammatically depict the two additional Hydro Photo Cell systems 130 and 130′ mentioned above, which utilize the principles of the present invention. To the extent systems 130 and 130′ comprise components, which are shown in FIGS. 6 and 7 and described above, no further description thereof will be given.

Specifically, system 130 of FIG. 8 is very similar to system 80 of FIG. 6, except system 130 comprises a light-transmitting tank 108′, which functions not only as a site for photosynthetic growth of the alga, but as a site where sparging also occurs. By combining the photosynthesis and sparging functions at a single site, effective dwell time of the alga in the photosynthetic zone of the system becomes 100% in the tank 108′. A vent or automatic relief valve 120′ is positioned at the top of closed tank 108′ to vent to the atmosphere the oxygen, derived from photosynthesis, which is displaced to the vent 120′ by bubbles of sparging air. Heat exchange 113 is diagrammatically illustrated in FIG. 8 as being incorporated into tank 108′, to maintain the liquid therein at the proper temperature.

Tank 108′ may be in the form of tank 108″, shown in FIGS. 9 through 11, with heat exchanger 113 centrally and longitudinally disposed therein. Note the length L is shown (in FIG. 9) to be much greater than the height H (FIG. 10), which is much greater than the width W (FIG. 10). This arrangement ensures substantially uniform and extensive exposure of the liquid within the tank 108″ to generally oppositely-directed light issuing from sources 90 and 92, located on opposite sides of the tank 108″. See FIG. 10. Tank 108″ is, therefore, elongated and rectangular, as shown. It is sealed to prevent entry of contamination.

The technology of FIGS. 9 and 10 otherwise comprises components substantially the same as explained above in conjunction with FIGS. 6 and 7. While shown diagrammatically only in FIG. 11, in most applications of the present technology, the pH, oxygen content and temperature of the liquid will be monitored, either continuously or periodically, typically at the alga growth sites 108, 108′, 108″ and 110.

As best shown in FIG. 9, CO₂ from source 84 and compressed air from source 86 are delivered in desired amounts across control 118 to a sparging line 132. Line 132 is uniformly porous. Desired amounts of CO₂ and compressed air are bubbled via porous line 132 into the liquid in tank 108″. The CO₂ supports photosynthesis, while the compressed air purges oxygen released during photosynthesis from the liquid in the tank for release via vent 120′.

Optimal growth of any isolated alga is a function of many factors, as explained to some degree above, including choice of nutrients, rates of flow of the liquid, the carbon dioxide, the nutrients, the addition of more alga and the removal of concentrated alga, the nature and amount of light, the type of algal growth receptacle and the dwell or residence time of the liquid therein, the nature and extent of sparging, the type of pumping and rates of pumping displacement and concentrations of the various additives. These variables can be reasonably set, controlled and adjusted by those skilled in the art for acceptable, if not optimal results.

Monitoring of important characteristics of the liquid and chemical analysis of the medium, as the algal culture grows, is helpful. Use of a state-of-the-art computer-based data acquisition and process control system is preferred. This allows both monitoring and control of the culture conditions continuously (and remotely by modem). It also accommodates expansion to handle increased production. Computer control of culture conditions greatly facilitate periodic adjustments to, for example, ammonium, pH, carbon dioxide, and bicarbonate concentrations, which involve interactions that can cause instabilities in culture conditions and be very deleterious if not quickly detected and corrected. Light levels on high-density cultures are a factor. Light availability may be the ultimate limiting factor involving growth in cultures. Availability of sunlight in conjunction with artificial light may be an enhancing combination. In some applications sunlight may be sufficient.

Preferably tanks used in the system embodying the present invention are made of acrylic. Acrylic is virtually as transparent to light as glass. The thinner water columns, formed by each tank, the better the light penetration. On the other hand, the thicker water column, the greater the volume of the tank which results in less light penetration. The greater the water volume, the more the production output, but the slower the growth due to reduced light.

There is preferably only gentle circulation of liquid in the tanks. This is to avoid cell damage that can be caused by violent agitation (shear). For example, AFA is shear sensitive.

To some degree, tanks replicate conditions in a lake or pond, but the cultivated alga is exposed to more uniform light in the presence of controlled nutrients and has restricted motion.

Tanks are typically fabricated as a single piece to provide a closed component of the closed system. Thus, size is limited by certain factors, such as transportation after fabrication, ease of placement, etc. In a practical sense, such factors may limit tank size to a width of about 20 feet, a height of about four (4) feet and a water column thickness of about six (6) inches.

As stated above, oxygen is generated, as a normal by-product of photosynthesis. In any one of the systems of FIGS. 6 through 11, the alga is grown in a closed system, with very high rates of growth. Therefore, much higher rates of oxygen generation occur than would be true for a lake or pond. This requires removal of oxygen, rather than allowing it to passively escape from solution. If the concentration of oxygen rises excessively in the presence of bright light, it becomes toxic to the alga.

After the alga travels through the bands of tubing, it exits, for example, into an output manifold, as shown in FIGS. 6 and 7. From there, it is displaced into a sparging tank or manifold. Sparging is the process of bubbling compressed air through the liquid (comprising algal product), usually immediately after it comes out of the tubes.

The alga is continually recirculating, to limit exposure to light in the tubes for a predetermined length of time depending on pump displacement, rate of algal growth, etc. In some embodiments, about 12 minutes is an appropriate residence time in an algal growth receptacle, such as tubes. This approach avoids buildup of oxygen within the tubing as a result of photosynthetic activity. The liquid is then emptied into the output manifold and sparged, to vent excess oxygen. After sparging, the liquid is pumped back into the input manifold and the process begins anew.

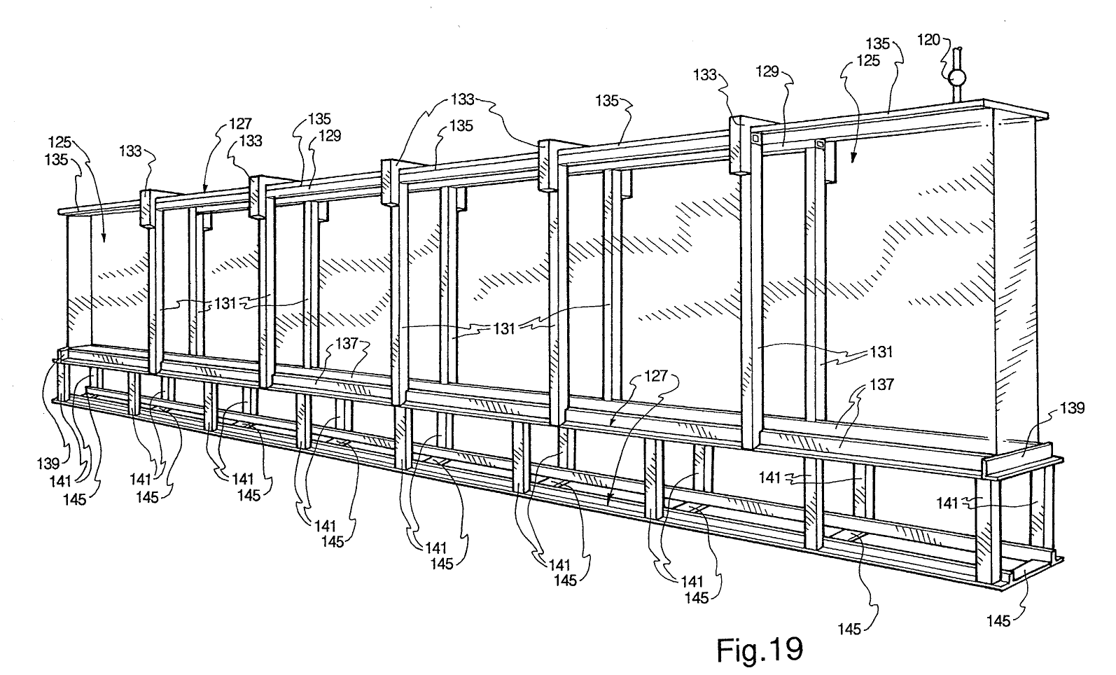

Reference is now made to FIG. 19, which illustrates a tall, narrow and longitudinally-elongated algal growth tank, generally designated 125. The tank 125 is formed of light transmitting material, preferably formed from a suitable synthetic resinous material, such as medical or food grade acrylic. The tank may comprise acrylic sheets, welded or bonded at all corners to form a closed container, except for influent alga/nutrient liquid, CO₂ and compressed air, effluent alga/nutrient liquid and with gas venting and provision for access panels.

An array of lights (not shown) is located along each elongated side of the tank 125 to promote photosynthesis. The tank 125 is supported against material tank component displacement by a framework, generally designated 127. The components or members of the framework 127 are connected by welding or with conventional fasteners. Framework 127 is constructed so as to minimize interference with light passing into the tank. To this end, a top beam 129 is provided contiguous with the tank near each of the two upper corners of the tank. The two top beams are supported upon spaced columns 131. The two top beams and each aligned pair of columns 131 are further held in position by top U-shaped brackets 133. Releasible sealed access top panels 135 are located between the beams 129 as may be some permanently sealed top tank wall segments.

The lower end of each column 131 connects with one or the other of two lower tank beams 137. Beams 137 are connected together at their respective ends and at intermediate locations as well by cross braces 139.

The described support framework 127 prevents distortion of deflection of the side walls of the tank 125 due to the force of the liquid being displaced therethrough under pressure. While other sizes may be used, tank 125 may be 48 inches high, six (6) inches wide and 20 to 30 feet long.

The bottom wall of the tank 125 is located, as illustrated in FIG. 19, well above the floor or other support surface, to provide access for inspection of the bottom of the tank. Thus, beams 137 are held in parallel, horizontal relation a desired distance above the floor by spaced short columns 141. The upper end of each column 141 connects to one or the other beam 137, while the lower end of each column 141 connects to one or the other of two floor beams 143. The predetermined spacing between parallel beams 143 is maintained by cross bars 145, each of which is connected to both beams 143.

### Sparging Tank

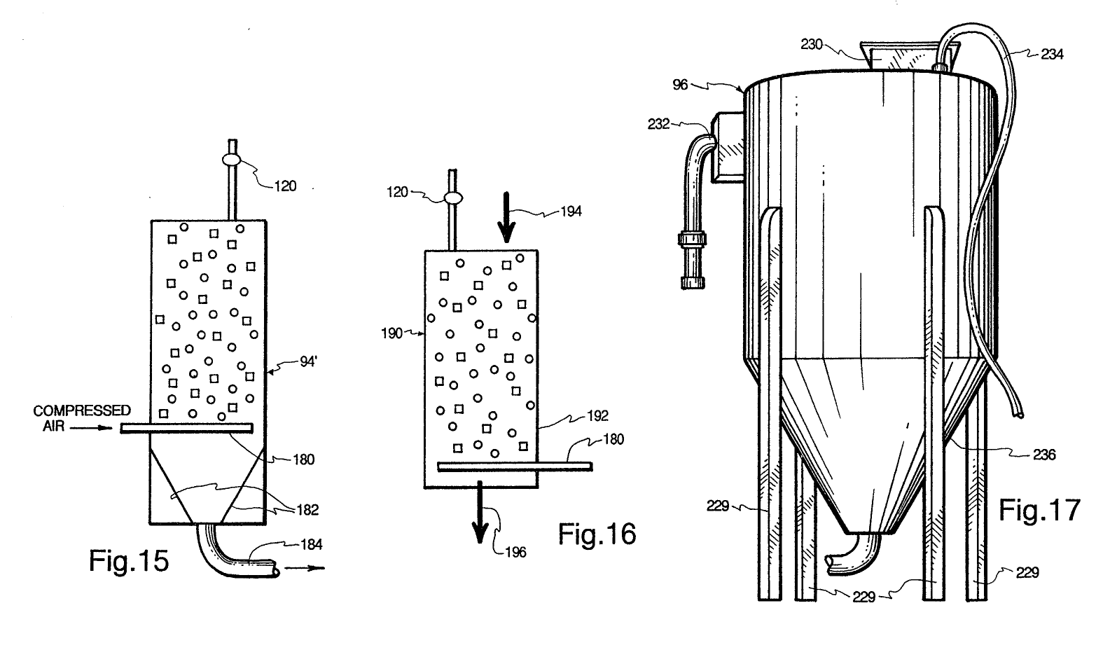

Reference is now made to FIG. 15, which diagrammatically illustrates one embodiment of a sparging tank, i.e., tank 94′. Tank 94′ comprises a large auxiliary tank connected to the Hydro Photo Cell (HPC) or algal growth receptacle 108 or tubes 110. Tank 94′ functions to receive as its influent the effluent from receptacle 108 or tubes 110 and to vent excess oxygen (sparging) through vent 120 by bubbling compressed air through the tank 94′. Compressed air is delivered from a compressor or other source to one or more sparging rods 180. Each sparging rod 180 comprises a hollow porous rod such that the compressed air is displaced through the center of the rod 180 and thence into the interior of the sealed tank 94′. As the air passes through the rod 180 it forms bubbles which rise through the liquid to the top of the tank.

The cone-shaped bottom 182 assists in directing flow of liquid from the closed system tank 94′ through pipe 184 to the pump 100.

Tank 94′ is also used for introduction of a regulated amount of CO₂ into the system to enhance photosynthesis and for control of pH, which will rise as a result of photosynthesis. In general, an excessively high pH restricts growth. Tank 94′ maybe of light-transmitting material to accommodate continued growth of alga therein.

In lieu of tank 94′, one or more oversized manifolds 190 (shown in FIG. 16), comprising one or more transparent tubes 192, may be used for sparging. Influent is displaced into the sparging manifold 190 at site 194, which may comprise a suitable valve. Effluent is displaced out the bottom at site 196, which may comprise a valve. Sparging manifold or pipe 190 may be four (4) to six (6) inches in diameter and comprise clear, food grade PVC. Sparging rod 180 functions as described above. Use of one or more manifolds 190 in lieu of a sparging tank provides for smaller liquid volumes compared to a tank and for better and more uniform exposure to light of the culture being circulated. Transparent materials, such as transparent tubing, are preferred because such also allows human visual observation of material inside.

In terms of its fundamentals, sparging comprises continuous input of compressed, filtered, aseptic air. For a 730 liter HPC, for example, 8 liters of sparging air per minute, at 10 psi comprises an appropriate input. In addition to sparging, this air input also maintains a positive air pressure in the system, to assist in preventing entry of contaminants. While other amounts could be used, typically the volume of liquid in tank 94′ will be about 5-10% by volume of the liquid within the entire closed system.

The sparging operation typically also involves injecting CO₂ so that the influent compressed air can be mixed with the CO₂ so as to reduce the shock caused by CO₂ contact with the culture comprising AFA or other species of algae as it enters the system or shortly thereafter. CO₂ should be introduced into the system so that it is not immediately lost by being prematurely vented, with or without excess oxygen and compressed air. Introduction just ahead of the supply or influent manifold, i.e., immediately prior to the pump, using pump action to help mix CO₂ into the liquid, prevents such premature venting. Introduction at other locations may also be appropriate.

A vent, such as vent 120 is disposed on top of the sparging tank 94′, to accommodate discharge of photosynthetic oxygen and sparging air. The entire system is closed, including the tank 94′, so that gas cannot enter except where selectively permitted. Accordingly, when the system is drained, for cleaning, repair or some other purpose, creation of a vacuum and fracturing some part of the equipment from resulting pressure should be avoided. This is done by opening the vents to microbe-free influent air, to place the interior of the system at atmospheric pressure.

When a sparging manifold is used, one or more sparging manifold vents will be included. Each vent typically comprises a filtered relief valve which excludes entry of airborne bacteria or other contaminants when, for example, air must be admitted as liquid comprising mature alga and media is withdrawn (harvesting).

In the case of a combination alga growth and sparging tank (such as 108″, shown in FIG. 9), a relief valve vent 120′ is provided for the purpose mentioned immediately above.

### The Growth Tubing and Framework in Combination

As stated above, the algal growth receptacle 108 (FIG. 6) may comprise a series of tubes 110 (FIG. 7). While the tubes 110 may be disposed in a linear array, helical arrays are currently preferred for reasons set forth below. Oval and other configurations for the tubing could be used. While eight coils of tubing 110 are illustrated in FIG. 7, any desired number may be used. Each tube 110 is light-transmitting and is preferably formed of clear polyvinyl chloride (PVC) food grade tubing to collectively circulate liquid comprising media and the AFA or other algal strain being cultivated so as to provide substantially uniform exposure of the liquid being circulated to a selected amount of controlled light issuing from sources 90 and 92 appropriate for effective photosynthesis. Trace minerals are added as specific nutrients to support the algal growth.

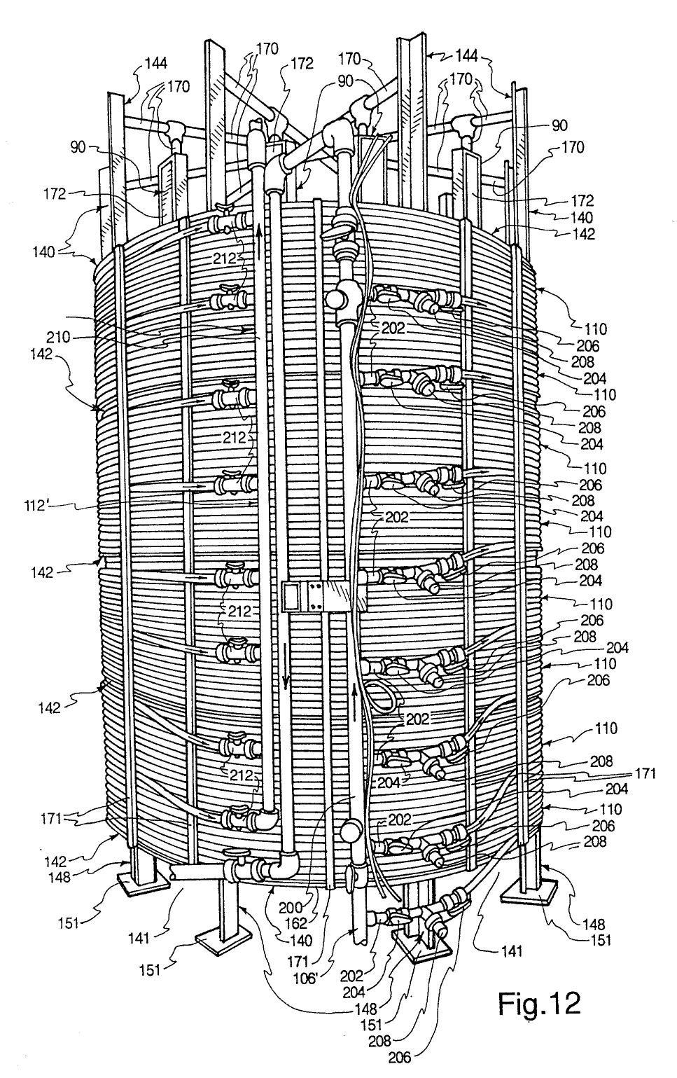

Reference is now to FIGS. 12 through 14, which depict one form of a helical array of tubes 110 coiled upon a cylindrical support framework. Specifically, the helical tubes 110 are supported in their vertical orientation upon a cylindrical stand or framework, generally designated 140. More than one framework 140 may be used, depending upon the number of coils of tubes 110 appropriate for a given operation. Each cylindrical framework 140 may be, for example, 30 feet high and 35 feet in diameter, although other sizes may be used as deemed appropriate by those skilled in the art.

The interior of the cylindrical framework 140 is shown as being open or generally hollow, for ease of access, among other things, although vertical supports or columns, beyond that which is shown in FIGS. 12 through 14, and/or cross-bracing within the framework 140 can be provided where additional strength is required.

The make-up of the framework 140 is illustrated as being modular, i.e., a series of substantially identical cylindrical sections 142 stacked vertically one on top of another, although a non-modular frame could be used. The top section 142, however, has short columns 144, comprising structural angles, extending vertically beyond the upper cylindrical edge 146 of top section 142, while the bottom section 142 has legs 148 extending below the bottom cylindrical edge 150. Each leg is illustrated as comprising a floor-engaging distal pedestal 151.

Otherwise, each cylindrical framework section 142 comprises a cylindrically-disposed sheet of expanded metal or grating 152, against which two coils of tubing 110 are tightly wrapped. One wrap of tubing is vertically disposed above the next.

Before the tubing is wrapped, each cylindrical sheet of expanded metal 152, when assembled, is essentially externally unencumbered (except for certain tubing supports explained below) and is internally supported. The internal support comprises a top annular or ring-shaped length of angle iron 154 welded to the interior of the associated sheet 152 and having one leg 156 in a horizontal plane extending inwardly toward the center of the cylindrical framework 140 and a second annular leg 158 extending downwardly. The internal support for each section 142 also comprises a bottom annular or ring-shaped inverted tee section 160 comprising one leg 162 in a horizontal plane extending both inwardly and outwardly toward and away from the center of the framework 140. The portion of the leg 162, which is directed outwardly, supports two vertically-stacked coils of the tubing 110. The inverted tee section 160 also comprises an upwardly directed annular leg 164, which is welded to the interior of the associated cylindrical sheet 152. The leg 162 of one section 142 may simply rest upon the next lower flange 146. However, clamping, fastening or welding of one section 142 to the next section 142 is preferred.

The internal support also comprises an annular horizontally-directed flange or rib 166 welded at its outside edge to the interior of the sheet 152, along the cylindrical midpoint thereof. Spaced vertical bars 168 extend between angle iron ring 154 and inverted tee section 160 and are welded to the interior of the sheet 152.

Each framework section or ring 142 is illustrated as supporting two bands, coils or segments of coiled tubing, each of which has multiple vertically stacked helical wraps. In one embodiment, each band 110 comprises about 430 linear feet of tubing, the primary limitation being the requirement for aeration to enhance photosynthesis without reaching the point of oxygen toxicity. Restricting the length of each tube 110 also reduces the pressure necessary for pumping.

The expanded metal sheets or grating 152 of sections 142 are 80% open to light, from inside the framework 140. This is important for effective use of artificial light from source 90 (FIG. 7) placed centrally within the framework 140 at sites 172.

Each ring 142 thus comprises an external annular base plate at flange 162 to support the first wrap of tubing 110. Each successive wrap is supported upon the previous wrap. This helps to maintain the helical configuration needed for tubing and to provide a stable platform for wrapping.

The diameter of framework 140 is proportional to the residence time required for algal growth, without incurring oxygen toxicity. The diameter may be selected to match even equal multiples of tubing wraps in the form of a helical band (e.g., 4 wraps per band, 5 wraps per band, etc.) to provide the correct toxogenic dwell or residence time. In one configuration, a framework 30 feet high and 35 feet in diameter is suitable.

The spaced short columns 144, shown in the form of angle irons, are welded at their lower ends to the top flange 146 of the top section 142 and serve to provide structural support for the source 90 of inside light and the wiring and wiring conduit.

The spaced legs 148 are welded at their top ends to the bottom flange 162 of the bottom section 142. Legs 148 support the weight of framework 140 and the tubing 110 upon pedestals 151 and create a lower spacer 141 for access through the space above a floor comprising, for example, a concrete pad engaged by each pedestal 151. This permits easy access to the center of the framework for maintenance, etc. and avoids potential safety problems related to “confined space.”

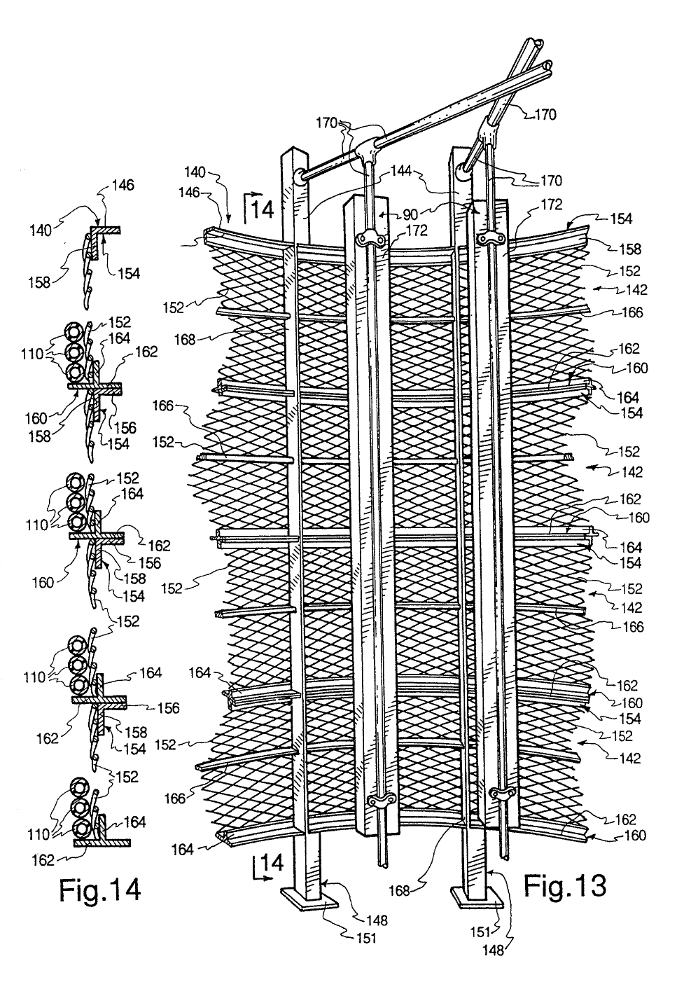

As shown best in FIGS. 12 and 13, a network of light-weight conduit 170 with wiring therein is connected to and supported by columns 144. The inside light source 90 is illustrated as comprising spaced vertically elongated lamps 172. The number and nature of the lamps 172 may vary as appropriately determined by those skilled in the art. The wiring within the conduits 170 supplies electrical energy to lamps 172 in a conventional way. While not shown in FIGS. 12 through 14, an array of lights will also be located on the outside of the coiled tubes 110.

### The Tubing

Each tube 110 preferably comprises transparent PVC tubing of a predetermined size and adequate to accommodate the desired rate and volume of displacement and necessary light transparency to support photosynthesis.

In one embodiment, PVC tubing of 1.0 inch inside diameter and 0.15 inch wall thickness is suitable, although other sizes will work. Generally, the larger the diameter the greater the capacity, up to the point where photosynthesis is negatively affected within the tubes 110 by reduced light transmittance and availability due to the size increase.

It is preferred to wrap tubing 110 under tension tightly around the cell so that each wrap is directly vertically over the one below. The tension in the wrapped tubing 110 pulls the tubing 110 radially inwardly against the cylindrical grating 152 to retain the successive wraps in taut vertically retained relationship. This assures uniform light availability to the interior of each wrap and avoids any shading effect. It is also more aesthetically pleasing to the human eye.

Preferably, the tubing 110 is not tied to the frame, but is held in place by the wraps, placed under tension (stretched), around the cylindrical grating 152, except for the one wrap of tubing, which rests on flange 162. The wraps of tubing 110 are also held in place by spaced vertical retainer bars 171 extending top to bottom on the outside of, but contiguous with, the tubes 110. The bars 171 are conventionally fastened in any suitable way to the framework 140 at selected locations.

As mentioned above, to assure maximum light availability to the alga being circulated in the coils of the tubing 110, very high clarity PVC tubing is suitable. Food grade PVC tubing, made from FDA- and NSF-approved materials, is usually preferred, because of the hydroponic type process involved, particularly where the end product 124 being generated comprises a nutritional food supplement for human consumption or an ingredient for a cosmetic.

The tubing 110 allows substantial light transmission through the tubing wall while providing “stacking” strength as a consequence of the tubes being placed on top of each other, without causing tube distortion and/or shading. Distortion and shading affects light transmission, which in turn have a negative affect on the growth rate of the alga. For example, if the tube 110 is compressed into an oval shape, it extends the distance through which some of the light must travel to reach all of the product being displaced therethrough. An oval shape also results in a shading effect to the tubes above and below. Where pig cleaning is contemplated, tubing with a circular cross-section is important.

Since the process depends on photosynthesis, maximum clarity and roundness of the tubing assures as much light transmission as possible for the alga inside the tubing.

It is recommended that the bands of tubing 110 be without joints or splices. Joints present difficulties in cleaning, using a pig, as explained hereinafter, and comprise possible entry points or refugia for contaminants.

### The Influent Manifold

The manifold 106 (FIGS. 6 and 7) is used to introduce the alga and nutrients or medium into tubing 110. One or more disinfectants may also be so introduced through the influent manifold, when it is advisable to do so. There is one other manifold, i.e., effluent manifold 112. For cleaning, a pigging manifold may be used. One suitable pigging manifold is described later in this specification. Also, as stated above, a sparging manifold may be used in lieu of a sparging tank.

The manifold comprising tubes 110 relieves input head pressure by subdividing the influent flow into several streams, one in each tube 110. For example, with Genesis 00 (15 feet high), pressure is normally 5 psi at the top and 7 psi at the bottom; a very small differential. To pump 730 liters, a typical amount for the system, through a single length of tubing having a cumulative length of 3,200 feet would require extremely high pressure and a powerful pump to overcome the head pressure differential, as well as the friction within the tubing, as well as normal air pressure. Extreme pressures may result in damage to the equipment, such as valves and joints. Thus, the separation of the influent into several streams, one in each tube 110, avoids excessive, potentially destructive pressures.

Manifold 106 may take the form illustrated in FIG. 12, at 106′. Manifold 106′ comprises an influent distribution pipe 200 and eight distribution pipes 202. The liquid in pipe 200 is obtained under positive pressure of pump 100 from sparging tank 94. An intake valve 204 is located in tube 200 upstream of the distribution pipes 202 to accommodate fill or partial shut-off of influent flow when and to the extent necessary for repairs and the like. As mentioned above, manifold 106′ subdivides a single stream into many streams thereby reducing the pump pressure required to displace the alga/media liquid.

Each pipe 202 connects with one of the tubes 110 across two flow control valves 204 and 206. A pig influent mechanism or pig insertion valve 208 is interposed between each set of valves 204 and 206.

One form of discharge, collection or effluent manifold is shown in FIG. 12 at 112′. The discharge end of each coiled tube 110 empties into a collection pipe 210 across a control valve 212. Each valve 212 may be closed, for repairs for example, fully opened or partially open as appropriate under a given set of circumstances.

Liquid collected in pipe 210 is displaced to sparging tank 94 across heat exchanger 113.

If desired, more than one manifold 106′ may be used, together with additional arrays of tubes 110.

### The Pump/Flow of Liquid

In regard to certain algae, such as AFA, displacement of the liquid comprising alga and nutrients can be very important. The nature, construction and operation of pump 100 is important for the same reason.

A dual lobe, positive displacement pump (e.g., Waukesha Model 130 PD) is used, pumping from the bottom up. No header tank is used. Pump clearance dimensions listed below allow the alga to pass with close tolerance, but without damage, yet still allowing efficient pump operation. Clearances between walls of the pump and inner parts is critical and is as follows:

From tip of lobes to inner walls, 0.0065-0.0085 inches.

From lobe to back wall of pump, 0.003-0.0035 inches.

From lobe to front cover plate, 0.010-0.015 inches. If clearance is materially less, the pump will likely damage the alga by crushing. If more, the pump must be run at higher speeds, to compensate for less effective flow. Higher speeds create turbulence, which also damages the alga. Minimum lobe-to-wall clearance in the normal direction of flow must be greater than the diameter of the AFA filaments. If this dimension is less than about 0.005 inches, continuous, cumulative alga damage to the alga will occur.

### Harvesting

Harvesting is preferably by continually pumping a nutrient mix from supply 88 (FIGS. 6, 7 and 8) into the algal growth receptacle 108 or tubes 110 or tank 108′ and withdrawing some of the liquid comprising concentrated alga into collection tank 96. The excess volume may be caused to overflow into the collection tank 96. A suitable form of tank 96 is illustrated in FIG. 17. The collection tank 96 may comprise a cone-shaped bottom 236, to collect settled and perhaps more concentrated biomass, which is removed from the tank 96 and processed in known ways, at 122, to arrive at a suitable product, at 124.

The collection tank 96 of FIG. 17 is illustrated as being supported upon legs 229 and has a fixed cover, which comprises a normally closed top access opening 230 and a sanitary one-way overflow 232, preventing contaminants from entering the tank. The tank 96 also comprises a compressed air injection tube 234 to aerate the alga and provide positive air pressure in the system. The system may also comprise a second holding tank, to temporarily store biomass. During this storage, the alga converts some mass into protein, possibly also beta carotene or other carotenoids. Use of the secondary holding tank accommodates control of the attributes of the contents thereof by selectively controlling light and aeration, resulting in a variation in the product or products.

### Controllers and Monitoring

Preferably, temperature monitors, pH controllers, dissolved oxygen monitor and conductivity monitors (see FIG. 13) are used. Data from these sensors are tracked by a computer controlled data acquisition and process control system (see FIG. 5) for analysis. These sensors not only help control the process, but also provide information on changes, or transformations in the product. The data acquisitions system accommodates printing of a record of the data so sensed and may be represented graphically, as well as used to predict changes or to analyze problems or to modify any of the variables associated with the system. Thus, the sensing and control system provides for precise operation on a remote basis.

The computer control system illustrated diagrammatically in FIG. 5 is a state-of-the-art system by which data from the pH, temperature, oxygen, conductivity, ORP and/or other sensors are input across a signal conditioning module 80 into a data acquisition board 82 into a personal computer (PC) 84. Control signals are issued from the PC 84 across the data control board 82 through module 80 to control the flow of carbon dioxide, the degree of heating at the heat exchanger, the amount and intensity of light and/or the input of nutrients. Displays, printers and other ancillary components of a conventional nature may be added to the computer control system.

### Water Supply

Use of a reverse osmosis filtering system is preferred, to remove micro-organisms, salts, etc. from the influent supply water to the nutrient supply site 88. Water from spent medium is preferably recycled. UV light is preferably used to eliminate micro-organisms after the water is filtered and after mixing of nutrients, etc. before reuse.

### Light

Use of artificial light is preferred to accommodate up to twenty-four-hour-a-day production. In some configurations, natural light with or without artificial light may be sufficient. Preferably the alga/nutrient mix in the closed system is exposed to light continuously or almost continuously (24 hours, every day). Essentially the only time alga may not receive light is during sparging, which may be in 12-minute cycles, when coils of tubing 110 are used. If clear manifolds are used for sparging, some light will be available even during sparging. Use of artificial light, of any suitable type, provides a substantial improvement over growth of algae depending exclusively on sunlight. Growth is maximized when under continuous light, whatever the source. Although artificial light may be less effective than natural sunlight in supporting algal growth, it can be utilized to maximize growth during low light periods ( i.e., at night or on cloudy days), especially when used in conjunction with sunlight. Light intensity is altered as one way to control growth. This is done by use of lights placed or positioned at varying distances. Florescent lights and high output metal halide lights are normally suitable sources of artificial light. Darkness causes cessation of growth.

### Nutrient Supply

Preferably a concentrated nutrient mix is combined with purified water to form the nutrient medium. The nutrient medium is displaced from source 88 to the sparging tank or manifold 94, across valve 114. The nutrient medium is UV light treated or filtered en route to the algal growth receptacle 108, the tubes 110 or the tank 108″, as an additional anti-microbial treatment. Typically, a unique blend of minerals is used for nutrients, depending on the alga being cultivated. By controlling the ratio of trace minerals, algal growth is sustained and some attributes of the alga are controlled. For example, proper control of cobalt in the nutrients will maximize the availability of vitamin B-12. By using an ammonium compound, nitrogen is supplied to the alga and conductivity of the media is controlled. Continuous addition of sodium nitrate results in an increase in mineral salts, which increases conductivity. High levels of mineral salts, however, appear to inhibit growth.

### Cleaning

Periodic cleaning of the interior of the system is a matter of routine maintenance. To clean the tanks, each tank is placed at atmospheric pressure and drained, the lids or closures to access openings are removed, and the interior cleaned with sponges, and suitable cleaning supplies. The tank is open, and, cleaning is therefore, simple.

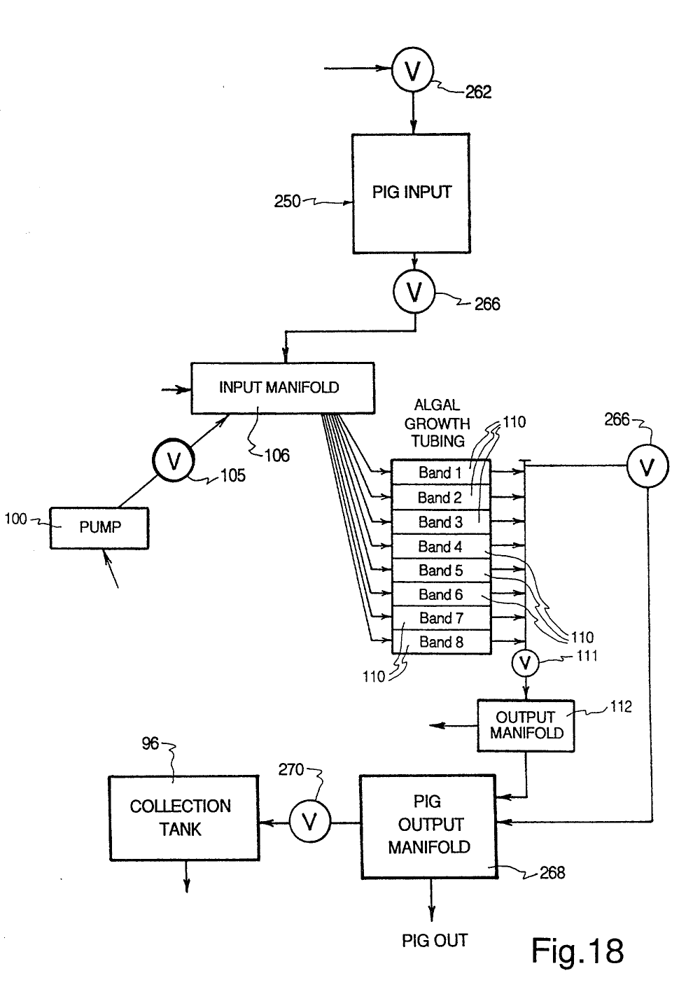

Periodically, tubes in all sections may need to be cleaned. Cleaning of pipes and tubes is preferably by “pigging” operation. For example, in reference to FIG. 18, a pig input, generally designated 250, is provided in conjunction with the discharge side of influent manifold 106′. A “pig” comprising a small piece of foam polymer or other suitable material having a uniform diameter may be used, which is pushed through the entire tube using water or air pressure. This is another reason for using round tubing having a predetermined diameter. The pig is sanitized (sterilized) prior to being placed into the system at input 250.

In the embodiment of FIG. 18, the pig is inserted into the sterile pig input manifold 250 and is placed under water or air pressure obtained from source across open valve 262, with pump 100 off and valve 105 closed. Valve 111 is open and valve 266 closed, if manifold 112 is to be cleaned. Otherwise, valve 111 is closed and valve 266 open. The pig passes into the input manifold 106′ and through one coil of tubing 110 during each run and its associated valves 204 and 206 (FIG. 12). The other tube influent valves 204 and 206 are closed. The pig, after traversing a selected tube 110, bypasses output manifold 112 and comes to rest in pig output manifold 268. The pig is removed from manifold 268. The used pig or a new one is inserted in pig input 250 and the cleaning process repeated until all of the tubes 110 have been consecutively cleaned. Valve 270 is open during the pig cleaning operation so that liquid delivered to the manifold is displaced across valve 270 to the collection tank 96.

Pig cleaning of tubes and pipes is a well-established art. Those skilled in the art may vary the manner in which pigging takes place, as is appropriate under the circumstances.

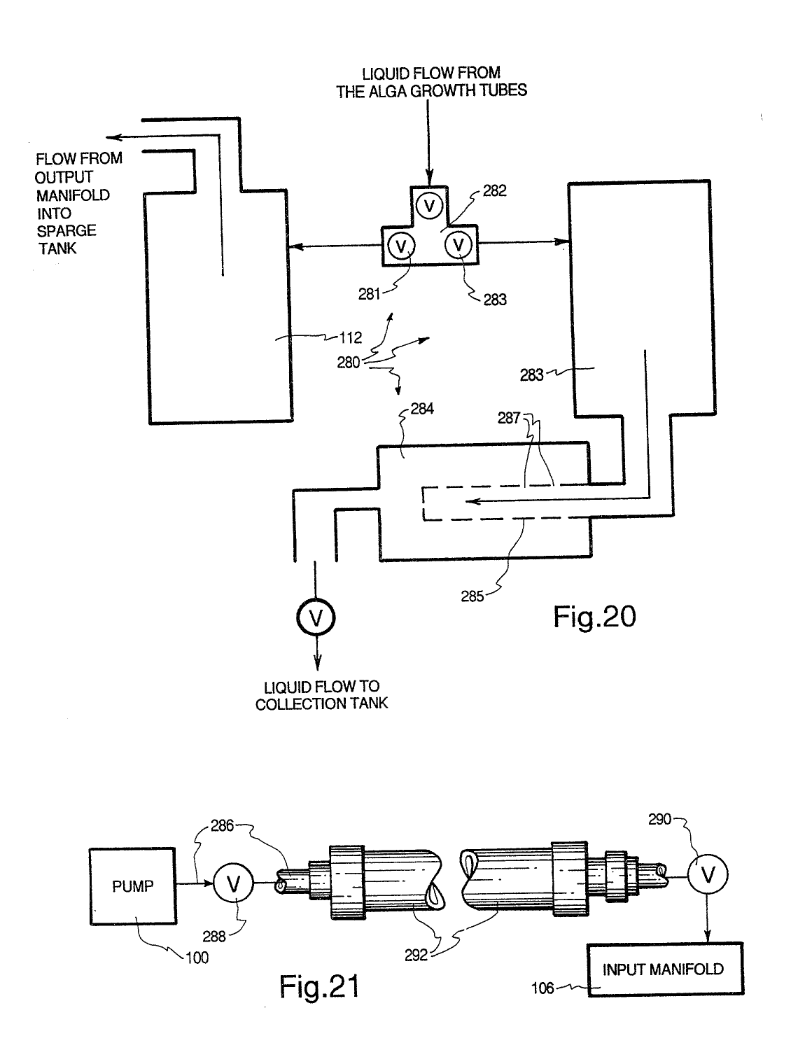

In the embodiment of FIG. 12, a pig is inserted into a selected insert valve 208 with associated valves 204 and 206 in closed position. After the pig is inserted at selected valve 208 and the valve 208 is sealed, valve 206 is opened. Compressed air is introduced via an insert to the cap of valve 208 and pushes the pig through one coiled tube 110. The pig passes through tube 110 through valve 212 and into a pig manifold. For pigging, flow through valve 212 is temporarily redirected to the pig manifold rather than into the output manifold 112. See FIG. 18. The pig is removed from pig catcher by disconnecting a valve at each end and reinserted (or a new pig is used) into another insert valve 208 in a sterile state and the cleaning process is repeated until all tubes 110 have been cleaned. Valve 270 (FIG. 18) is open during the pigging operation so that liquid delivered to the manifold is displaced across valve 270 to the collection tank 96. To remove pigs, valves 112 and 266 must be in closed position. The pig catcher 285 (FIG. 20) has connections at each end that allow the catcher 285 to be temporarily disconnected to allow removal of the pigs. The catcher 285 is then reconnected and valve 111 or 266 is moved to open position. Under normal operating conditions, the valve 266 remains in closed position. It is open only during those times when pigging is in process.

Reference is now made to FIG. 20 which diagrammatically illustrates a pig cleaning system, generally designated 280, comprising a three-way valve 282, a pig manifold 283 and a pig catcher 284, which may be used to clean the above-described tubes 110.

To clean one tube 110, the output manifold 112 and the heat exchanger 113, a pig is provided in combination with the triple manifold system 280. Tube cleaning takes place as described above. Removing the pig is done using the three-way valve 282 and temporarily diverting liquid flow emanating from the tube 110 being cleaned away from the output manifold 112 into the pigging manifold 283. As stated above, the pig is sanitized prior to being placed into the tubing.

The triple manifold cleaning system 280 serves to minimize the risk of introducing contaminants into the closed algal growth system.

Without this system, a pig would have to be inserted into one end of the tubing at the supply or influent manifold, flushed through and removed at the other end, by, for example, disconnecting a fitting at the output or effluent manifold. This creates a risk of contamination. Removing and reconnecting tubing at each manifold would also place undue strain on valves and connections.

The manifold system 280 allows for insertion and removal of a cleaning pig in a sanitary fashion. As shown in FIG. 21, the supply manifold is illustrated as comprising a by-pass line 286 between the pump 100 and the manifold 106.

The by-pass line 286 is equipped with two valves 288 and 290 located on opposite sides of an enlarged pig insertion chamber 292. When valves 288 and 290 are shut, the chamber 292 is opened by loosening threaded couplings 294 and 296 and the sterile pig is inserted into the chamber 292. The chamber 292 and couplings are reassembled and tightened and two valves 288 and 290 are then opened thereby displacing the pig through one selected tube 110. This process is repeated for each tube 110 by opening and closing selected valves 206 and 208.

As the pig approaches the output manifold 112, three-way valve 282 adjacent to the output manifold is adjusted to divert the pig into the “pigging” manifold 283. Once the pig passes into the pigging manifold the valve 282 is adjusted to allow fluid to again enter the output manifold and on to the sparging tank. The pig passes through the pigging manifold 283, is captured in a clear perforated tube 285 in the pig catcher 284.

The pig catcher 284 is a large diameter, clear PVC tube. It comprises a by-pass section of PVC pipe between the pig manifold and collection tank. It is used solely to capture and hold the pig. It has small diameter holes 287 to allow liquid to flow around the trapped pig on the way to the collection tank. It thus captures pigs without interfering with movement of liquid.

The trapped pig is removed from the pig catcher 284 by uncoupling the pig catcher 284 at one or both ends.

The invention may be embodied in other specific forms without departing from the spirit of the essential characteristics thereof. The present embodiments, therefore, are be considered in all respects as illustrative and are not restrictive, the scope of the invention being indicated by the appended claims rather than by the foregoing description, and all changes which come within the meaning and range of equivalency of the claims are therefore intended to be embraced therein.

What is claimed and desired to be secured by Letters Patent is:

1. A method of obtaining a supply of a desired alga comprising the acts of:
obtaining a multi-species sample of algae;
isolating the desired alga from the other algae;
cultivating the isolated alga causing the alga to grow.

2. A method according to claim 1 wherein the isolating act comprises microscopically separating the alga from the other algae.

3. A method according to claim 1 wherein the cultivating act comprises placing the isolated alga in a medium comprising at least one nutrient.

4. A method according to claim 1 wherein the cultivating act comprises circulating the isolated alga to promote growth and photosynthesis and oxygen removal.

5. A method according to claim 1 wherein the cultivating act comprises no flow and flow growth which takes place in a liquid growth media.

6. A method according to claim 1 further comprising the acts of inducing photosynthesis in the alga by use of light and sparging by which excess oxygen resulting from photosynthesis is removed.

7. A method according to claim 1 further comprising the acts of adding carbon dioxide and air to the alga in the presence of light to promote photosynthesis and to remove oxygen from the growth medium so that efficient photosynthesis is promoted.

8. A method according to claim 1 wherein the cultivating act takes place in a liquid medium displaced through an algal growth receptacle.

9. A method according to claim 1 wherein the cultivating act takes place in a liquid medium displaced in parallel through a plurality of transparent tubes in the presence of light.

10. A method according to claim 1 further comprising the acts of bifurcating the cultivated alga into a harvested portion and a separate root stock portion.

11. A method of producing an algal product comprising the acts of:
isolating at least one alga from an algae source;
providing nutrition in liquid form to at least one alga;
providing for photosynthetic growth in the liquid of at least one alga in the presence of light;
evacuating oxygen derived due to photosynthesis from the liquid to avoid oxygen toxicity;
harvesting, processing and productizing at least one alga.

12. A method of producing an alga comprising the acts of:
providing sources of alga, carbon dioxide under pressure, air, and at least one nutrient;
selectively introducing the alga, carbon dioxide under pressure, compressed air and the nutrient into a closed liquid flow system for cultivating the alga;
promoting photosynthesis of the alga in the presence of light, nutrient and carbon dioxide as the liquid is displaced in the closed system to cause algal growth;
sparging from the closed system oxygen derived during photosynthesis using the compressed air;
harvesting at least some of the mature alga from the closed system.

13. A method according to claim 12 wherein the mature alga is separated into a harvested portion and a root stock portion.

14. A method according to claim 13 wherein the separation of the mature alga is continuous.

15. A method of growing an algal comprising the acts of subjecting the alga to photosynthesis in the presence of nutritional media and carbon dioxide, venting oxygen derived during photosynthesis and harvesting the resulting alga.

16. A method of obtaining a supply of a selected alga comprising the acts of:
isolating the selected alga from a source comprising several species of algae and contaminants;
placing the isolated alga in a contamination-free environment;
growing the isolated alga into a concentrated form in the contamination-free environment;
harvesting the isolated and concentrated alga from the contamination-free environment.

17. Growing of one or more selected species of algae comprising the acts of:
subdividing a liquid stream of at least one alga, nutrients and carbon dioxide into a plurality of substreams;
displacing each substream through a light-transmitting tube in the presence of light to promote photosynthesis and algal growth;
removing oxygen produced by the photosynthetic activity of the alga;
processing at least some of the alga grown via the process of photosynthesis into product form.

18. A method of obtaining a pure single species alga comprising the acts of:
obtaining a quantity of liquid comprising a plurality of algae;
identifying a desired alga within the liquid and removing at least one single cell comprising the desired alga from the liquid;
placing the removed single cell in a growth medium and subjecting the single cell and medium to light to promote photosynthesis whereby alga growth takes place.

19. A method according to claim 17 wherein the alga is AFA.

20. A method according to claim 17 wherein the alga is Oocystis borgei.

21. A method according to claim 17 wherein the obtaining act comprises removing a liquid sample from a naturally-occurring source of water.

22. A method according to claim 21 wherein the obtaining act comprises removing a sample from a source and subdividing the sample .

23. A method according to claim 17 wherein the identifying act comprises use of a microscope.

24. A method according to claim 17 wherein the removing act comprises use of a small pipet or needle.

25. A method according to claim 17 wherein the placing act comprises placement of the single cell and the growth medium in a well or other container.

26. A method according to claim 17 wherein the placing act comprises transferring the alga and medium from a relatively small growth environment to a relatively large growth environment.

27. A method according to claim 17 further comprising the act of harvesting at least some of the grown alga.

28. A method according to claim 27 further comprising the act of processing and packaging harvested alga.

29. A method according to claim 17 wherein the placing act comprises creating and maintaining a contamination-free environment in which said algal growth occurs.

30. A method according to claim 17 wherein the placing act comprises verifying the absence of any alga other than the desired alga.

31. A method according to claim 30 wherein the verification is via a microscope.

32. A method of providing algal growth comprising the acts of:
providing a liquid stream comprising one or more species of algae and growth-promoting ingredients;
dividing the stream into a plurality of substreams;
subjecting each stream to light thereby promoting photosynthetic cultivated growth of the algae;
processing the cultivated algae into a suitable product form.

33. A method according to claim 32 wherein the providing act comprises commingling the algae from a first source, nutrients from a second source and carbon dioxide from a third source.

34. A method according to claim 33 wherein the commingling act comprises displacing the algae, the nutrients and the carbon dioxide under pressure.

35. A method according to claim 32 wherein the liquid is circulated between a plurality of sites while achieving the providing, dividing and subjecting acts and wherein the processing act is preceded by the act of removing only a portion of the cultivated algae from the circulation pattern.

36. A method according to claim 32 wherein the providing act is limited to a stream comprising one alga only.

37. A method according to claim 36 wherein the one alga is AFA.

38. A method according to claim 36 wherein the one alga is Oocystis borgei.

39. A method according to claim 36 wherein the one alga comprises an alga from the genus Haematococcus or the class Eustigmatophyceae.

40. A method according to claim 32 wherein the dividing act takes place in a manifold.

41. A method according to claim 32 wherein the dividing act directs the substreams into a plurality of transparent tubes.

42. A method according to claim 41 wherein the transparent tubes are vertically stacked in respect to each other.

43. A method according to claim 41 wherein at least some of the tubes are disposed in a coiled configuration.

44. A method according to claim 43 wherein the coiled configuration is vertically oriented.

45. A method according to claim 32 wherein the subjecting act comprises controlling the length of time photosynthesis occurs in the substreams and sparging oxygen, derived from photosynthesis, from the liquid to prevent toxicity

46. A method according to claim 32 further comprising the act of controlling the temperature of the liquid.

47. A method according to claim 32 further comprising the act of monitoring characteristics of the liquid.

48. A method according to claim 47 further comprising the act of adjusting the characteristics and/or environmental conditions of the liquid.

49. A method according to claim 48 wherein the adjusting act is computer-controlled.

50. A method according to claim 32 wherein the subjecting act comprises use of artificial light.

51. A method according to claim 32 wherein the subjecting act comprises use of natural light.

52. A method according to claim 32 wherein the providing, dividing and subjecting acts take place in a contamination-free closed system.

53. A method of promoting algal growth comprising the acts of:
providing a liquid stream comprising one or more species of algae and growth-promoting ingredients;
delivering the liquid stream to an algal growth site;
subjecting the liquid to light at the growth site thereby promoting photosynthetic cultivated growth of the algae;
processing the cultivated algae into a suitable product form.

54. A method according to claim 53 wherein the providing act comprises commingling the alga from a first source, nutrients from a second source and carbon dioxide from a third source.

55. A method according to claim 54 wherein the commingling act comprises displacing the alga, the nutrients and the carbon dioxide under pressure.

56. A method according to claim 53 wherein the liquid is circulated between a plurality of sites comprising the growth site among others while achieving the providing, delivering and subjecting acts and wherein the processing act is preceded by the act of removing only a portion of the cultivated algae from the circulation pattern while continuing to circulate the remaining portion.

57. A method according to claim 53 wherein the providing act is limited to a stream comprising one alga only.

58. A method according to claim 57 wherein the one alga is AFA.

59. A method according to claim 57 wherein the one alga is Oocystis borgei.

60. A method according to claim 57 wherein the one alga comprises an alga from the genus Haematococcus or the class Eustigmatophyceae.

61. A method according to claim 53 wherein the delivering act comprises displacing the stream into a receptacle at the algal growth site.

62. A method according to claim 53 wherein the delivering step comprises displacing the steam into a tank comprised of a light-transmitting material.

63. A method according to claim 53 further comprising the act of sparging oxygen, derived from photosynthesis, from the liquid to prevent toxicity.

64. A method according to claim 63 wherein the sparging act is practiced at the growth site.

65. A method according to claim 63 wherein the sparging act is practiced at a site downstream from the growth site.

66. A method according to claim 53 wherein the subjecting act comprises controlling the length of time photosynthesis occurs and sparging oxygen, derived from photosynthesis, from the liquid to prevent toxicity

67. A method according to claim 53 further comprising the act of controlling the temperature of the liquid.

68. A method according to claim 53 further comprising the act of monitoring characteristics of the liquid.

69. A method according to claim 68 further comprising the act of adjusting the characteristics and/or environmental conditions of the liquid.

70. A method according to claim 69 wherein the adjusting act is computer-controlled.

71. A method according to claim 53 wherein the subjecting act comprises use of artificial light.

72. A method according to claim 53 wherein the subjecting act comprises use of natural light.

73. A method according to claim 53 wherein the providing, delivering and subjecting acts take place in a contamination-free closed environment.

74. A system for algal growth comprising:
a source of at least one species of algae;
a source of carbon dioxide;
a source of air;
a source of at least one nutrient;
an illuminated algal photosynthetic growth site to which the alga, the carbon dioxide and the nutrient are delivered;
an oxygen sparging site where the air is delivered;
a sparged oxygen discharge site;
a collection site for grown alga.

75. A system according to claim 74 wherein the system is closed and contamination-free.

76. An apparatus for cultivating algal growth comprising:
at least one light-transmitting receptacle to which a liquid comprising an alga and nutrients and carbon dioxide are delivered;
at least one source of light juxtaposed and directed towards the receptacle to promote photosynthesis;
a compressed air injection device by which air is introduced into the liquid to sparge photosynthetically-derived oxygen from the liquid.

77. An apparatus according to claim 76 wherein the receptacle comprises at least one narrow, elongated tank comprised of light-transmitting material.

78. A method according to claim 76 wherein at least one receptacle comprises a plurality of tubes.

79. A method according to claim 78 wherein the tubes comprise a vertical array.

80. A method according to claim 78 wherein the tubes comprise a coiled vertical array.

81. A method according to claim 76 further comprising a cylindrical framework upon which a plurality of tubes are helically coiled in vertically stacked relation.

82. A method according to claim 81 wherein the framework comprises a hollow interior in which a plurality of light sources are located.

83. A method according to claim 81 wherein the framework comprises a perforated structural material accommodating transmission of light therethrough into the tubes.

84. An apparatus according to claim 83 wherein the tubes are helically wrapped against the perforated structural material.

85. An apparatus according to claim 83 wherein the framework comprises solid structural members to which the perforated structural material is connected

86. A method according to claim 76 wherein the receptacle comprises tubes supported by a cylindrical framework comprised of vertically-stacked modular subframeworks.

87. An apparatus according to claim 76 further comprising a cleaning assembly adjacent the receptacle whereby a pig can be introduced into the apparatus, displaced within the apparatus to clean, and removed from the apparatus.

88. An apparatus according to claim 87 wherein the cleaning assembly comprises a component whereby the risk of contamination in respect to cleaning is greatly alleviated.

89. A method of cultivating algae comprising commingling at least one alga and at least one nutrient, in liquid form; subjecting the liquid to light thereby promoting photosynthesis and controlling the residence time during which the liquid is exposed to light to prevent oxygen toxicity.

90. A method according to claim 89 further comprising infusing compressed air into the liquid during and/or after photosynthesis to purge at least some of the photosynthetically-derived oxygen from the liquid.
